(19) United States
(12) Patent Application Publication
(10) Pub. No.: US 2011/0003359 A1
(43) Pub. Date: Jan. 6, 2011

(54) BIODEVICE

(76) Inventors: Yoichi Fujiyama, Kyoto (JP); Yoichi Tagawa, Kanagawa (JP); Shunsuke Kobayashi, Kanagawa (JP)

Correspondence Address: Cheng Law Group, PLLC, 1100 17th Street, N.W., Suite 503, Washington, DC, 20036, US

(21) Appl. No.: 12/865,837

(22) PCT Filed: Feb. 3, 2009
(86) PCT No.: PCT/JP2009/051797

§ 371 Date: Aug. 2, 2010

(30) Foreign Application Priority Data
Feb. 4, 2008 (JP) 2008-023638

(51) Int. Cl. C12N 11/00

(52) U.S. Cl. 435/174

(57) ABSTRACT

Disclosed is a biodevice which has a porous membrane (8) and flow paths (10) and (12) formed therein. In a preferred embodiment, the flow path (10) and the flow path (12) are opposed to each other with the porous membrane (8) being interposed between them. The flow path (10) serves as a first reaction chamber through which a first solution is allowed to pass so as to be brought into contact with one of the surfaces of the porous membrane (8). The flow path (12) serves as a second reaction chamber through which a second solution is allowed to pass so as to be brought into contact with the other surface of the porous membrane (8). In the flow path (10), first cells are immobilized on the porous membrane (8). In the flow path (12), second cells are immobilized on the porous membrane (8).

### BACKGROUND OF THE INVENTION

1. Field of the Invention

The present invention relates to a biodevice for use as an artificial organ such as an artificial liver or for use in, for example, drug metabolism tests using cells.

2. Description of the Related Art

The liver is called the chemical factory of the body. It is currently known that the liver performs over 500 metabolic reactions. As one example of artificial organs, an artificial liver that can perform the functions of the liver is under development. However, it is very difficult to develop an artificial device capable of supporting all the functions of the liver without using living cells not only in the short term but also in the medium and long term, and therefore, it is believed that using living liver cells is the only way to develop an artificial liver. Such an artificial organ using living cells and an artificial device in combination is called a hybrid artificial organ. For example, a conventional artificial liver system for supporting liver functions uses liver tissues isolated from, for example, pigs (see Publication of unexamined application JPA 10-506806).

In one conventional artificial organ using living cells, a cell suspension containing cells and a cell culture medium is circulated to bring the cells into contact with blood or plasma separated from the cell suspension by a semi-permeable membrane (see JPA 10-506806). In another conventional artificial organ using living cells, a liquid is brought into contact with cells immobilized on the surface of or inside fibers or a porous membrane (see JPA 2006-296367). These conventional artificial organs use only one type of cells.

In the liver in vivo, hepatic parenchymal cells as well as sinusoidal endothelial cells etc. are regularly arranged. It is believed that signal transfer between these cells and circulation of body fluids play an important role in maintaining normal liver functions. Particularly, hepatic parenchymal cells and sinusoidal endothelial cells are arranged in rows. The rows of hepatic parenchymal cells are parallel to, but not in direct contact with, the rows of sinusoidal endothelial cells. The space between hepatic parenchymal cells and sinusoidal endothelial cells is called the space of Disse which contains an extracellular matrix. In the extracellular matrix, bile ducts are located on the hepatic parenchymal cell side and blood vessels are located on the sinusoidal endothelial cell side.

In order to build an artificial liver system having liver functions, it is important to regularly arrange hepatic parenchymal cells and sinusoidal endothelial cells and to form an extracellular matrix. Drugs and nutrients are supplied from blood vessels and then excreted into bile ducts. Attempts have been made to develop an artificial liver system using cultured cells, but an artificial system capable of stably performing liver functions for a long period of time has not yet been developed. This is because cultivation of liver cells is difficult per se, and formation of tissues similar to the liver in vivo is more difficult.

### SUMMARY OF THE INVENTION

It is therefore an object of the present invention to provide a biodevice using two types of living cells, which is capable of stably maintaining its function and can be used not only as an artificial organ such as an artificial liver but also for other purposes.

The biodevice according to the present invention uses a porous membrane as an alternative to an extracellular matrix. More specifically, two different types of cells are immobilized separately on two different surfaces of the porous membrane to perform signal transfer and exchange or transfer of materials between the two different types of cells via the porous membrane.

That is, the present invention is directed to a biodevice including: a porous membrane provided in a container and having two surfaces; a first reaction chamber in which a first solution is stored or through which it is allowed to flow so as to be brought into contact with one of the surfaces of the porous membrane; a second reaction chamber in which a second solution is stored or through which it is allowed to flow so as to be brought into contact with another surface of the porous membrane, the second reaction chamber being opposed to the first reaction chamber with the porous membrane being interposed between them; first cells immobilized on the porous membrane in the first reaction chamber; and second cells immobilized on the porous membrane in the second reaction chamber, the second cells being different from the first cells.

Examples of the porous membrane used in the present invention include polycarbonate track-etched membranes, PET (polyethylene terephthalate) track-etched membranes, cellulose-based porous membranes, nylon-based porous membranes, glass fiber porous membranes, polyether porous membranes, fluorine resin porous membranes, and ceramic-based porous membranes. An appropriate thickness of the porous membrane is several hundred micrometers or less. If the thickness of the porous membrane is larger than several hundred micrometers, the efficiency of material exchange and signal transfer between the first cells and the second cells via the porous membrane is reduced.

According to a preferred embodiment of the present invention, the biodevice is a chip-type device which has a porous membrane provided in a base body and having two surfaces, along each of which a solution is allowed to flow. In such a chip-type device, the porous membrane is provided in a base body, the first reaction chamber is formed by the base body as a flow path through which the first solution is allowed to flow along one of the surfaces of the porous membrane, and the second reaction chamber is also formed by the base body as a flow path through which the second solution is allowed to flow along the other surface of the porous membrane.

The flow paths as the first and second reaction chambers preferably have a depth of 1 mm or less. This is because these paths serve as a blood vessel or a ureter, and therefore, it is not necessary for the paths to have a depth greater than 1 mm. In addition, too great a depth of the paths leads to a disadvantageous reduction in concentration.

The surfaces of the porous membrane, on which the cells are immobilized, are preferably covered with a coating for cell culture. Examples of the material of the coating include gelatin, collagen, and biomaterials generally used as scaffolding for cells.

The biodevice according to the present invention can be used as an artificial organ. In this case, the first cells are, for example, hepatic parenchymal cells and the second cells are, for example, endothelial cells. The porous membrane functions as an alternative to the space of Disse, which makes it possible to regularly arrange the cells and achieve signal transfer between the cells. Further, the first and second solutions contained in the first and second reaction chambers located on opposite sides of the porous membrane function like blood and bile, respectively. Therefore, the biodevice according to the present invention has functions similar to those of an organ in vivo, such as metabolism and uptake and excretion of drugs.

According to the present invention, two different types of cells are immobilized separately on two different surfaces of a porous membrane. This makes it possible to easily form a structure similar to that in vivo which performs circulation of body fluids and signal transfer between cells.

Further, according to the present invention, it is possible to provide a device having flow paths in its base body. In such a device, cells can be cultivated in a space optimally designed for the cells and fluids are allowed to flow through the flow paths under control (e.g., fluids can be continuously fed to the flow paths or fluids flowing through the flow paths are caused to pulsate). Therefore, the use of the device according to the present invention makes it possible to conduct research on various systems and therefore to achieve an environment suitable for tissue formation.

### BRIEF DESCRIPTION OF THE DRAWINGS

FIG. 1 is a sectional view of Example 1 according to the present invention.

FIG. 2A is a plan view of an upper substrate of Example 2 according to the present invention.

FIG. 2B is a plan view of a lower substrate of Example 2 according to the present invention.

FIG. 3 is a sectional view of Example 3 according to the present invention.

FIG. 4 is a graph showing the results of an experiment to determine whether reaction containers of Example 3 and comparative examples perform liver functions.

### DETAILED DESCRIPTION OF THE INVENTION

### Example 1

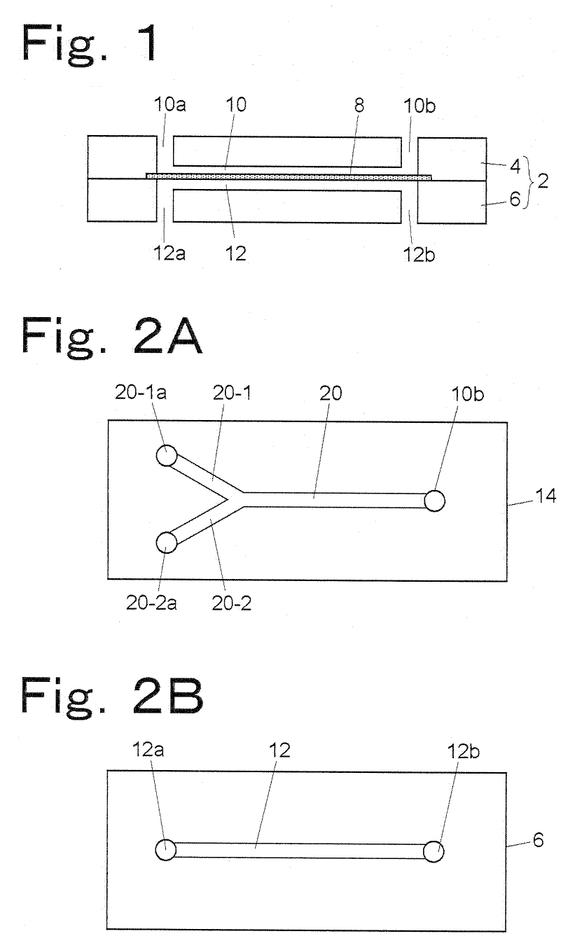

FIG. 1 is a sectional view of a chip-type device according to Example 1 of the present invention having flow paths, which is taken along the flow paths.

This biodevice is a chip structure including a base body 2 obtained by bonding substrates 4 and 6 together and a porous membrane 8 interposed between the substrates 4 and 6. The material of the substrates 4 and 6 is not particularly limited, but in this case, PDMS (polydimethylsiloxane) (“SILPOT184” manufactured by Dow Corning Toray Co., Ltd.) is used by way of example. The porous membrane 8 is not particularly limited either, but in this case, a mixed cellulose ester membrane filter having a thickness of 100 μm (“RAWP” manufactured by Millipore) is used.

The substrate 4 has a first flow path 10, a liquid inlet 10a, and a liquid outlet 10b. The first flow path 10 is provided along one of the surfaces of the porous membrane 8. The liquid inlet 10a and the liquid outlet 10b are provided at both ends of the flow path 10 so as to penetrate the substrate 4. The substrate 6 has a second flow path 12, a liquid inlet 12a, and a liquid outlet 12b. The second flow path 12 is provided along the other surface of the porous membrane 8 so as to be opposed to the first flow path 10. The liquid inlet 12a and the liquid outlet 12b are provided at both ends of the flow path 12 so as to penetrate the substrate 6. The flow paths 10 and 12 are grooves formed in the substrates 4 and 6 by molding, respectively, each of which has a width of about 1 mm, a depth of about 0.1 mm, and a length of about 20 mm. The liquid inlets 10a and 12a and the liquid outlets 10b and 12b are through-holes formed by subjecting the substrates 4 and 6 to through-hole processing.

In the first flow path 10, hepatic parenchymal cells are immobilized as first cells on the porous membrane 8, and cultivated. In the second flow path 12, endothelial cells are immobilized as second cells on the porous membrane 8, and cultivated.

A method for producing the biodevice according to Example 1 will be described. The substrate 4 having the flow path 10 and the through-holes 10a and 10b and the substrate 6 having the flow path 12 and the through-holes 12a and 12b are placed so that the flow path 10 and the flow path 12 are opposed to each other. Then, the substrates 4 and 6 are bonded together with the porous membrane 8 being interposed between them. In this way, a chip structure composed of the base body 2 obtained by bonding the substrates 4 and 6 together and the porous membrane 8 provided in the base body 2 is obtained. It is to be noted that the substrates 4 and 6 can be firmly bonded together by treating their surfaces to be bonded together with oxygen plasma.

Then, cells are immobilized on the porous membrane 8 and cultivated. However, before the cells are introduced into the chip structure, the chip structure is brought into a state suitable for cultivation of target cells in the following manner. First, the chip structure is sterilized by, for example, an autoclave, and then a biomaterial polymer such as gelatin is allowed to flow through the flow paths 10 and 12 to coat the inner surfaces of the flow paths 10 and 12 and the porous membrane 8 with the biomaterial polymer. Then, hepatic parenchymal cells are introduced into the flow path 10 through the liquid inlet 10a and endothelial cells are introduced into the flow path 12 through the liquid inlet 12a. These cells are immobilized on the porous membrane 8 and then cultivated to obtain a biodevice according to Example 1.

The step of immobilizing cells on the porous membrane 8 and cultivating the cells will be described in more detail.

(1) Gelatin Coating

0.5 g of gelatin is dissolved in 500 mL of pure water (Milli-Q water) and is placed in an autoclave at 121° C. and 2 atmospheres for 20 minutes to prepare a gelatin solution. The gelatin solution is introduced into the chip structure using a syringe and the chip structure is allowed to stand in a refrigerator at 4° C. for 2 hours or longer.

The gelatin solution remaining in the chip structure is removed before use. When cells to be immobilized on the porous membrane 8 are inoculated, a culture medium suitable for the growth of the cells is introduced into the chip structure.

(2) Procedure of Immobilizing Cells on Surfaces of Multilayer Membrane of Chip Structure

Cells cultivated on a Petri dish are washed with PBS (Phosphate Buffered Salts) three times and removed with a trypsin solution. Then, the cells removed from the Petri dish are suspended in a culture medium and centrifuged at 4° C. and 800 rpm for 5 minutes. The resultant supernatant is removed, and the cells are resuspended in a small amount of culture medium corresponding to the volume of the flow path 10 or 12 of the chip structure. The obtained cell suspension is introduced into the chip structure using a syringe, and the chip structure is allowed to stand in a 5% CO₂ incubator at 37° C. for 3 hours in a state where the porous membrane 8 is located under the cell suspension.

Cells are allowed to settle by gravitation and are then adhered to the porous membrane 8. Therefore, cells can be adhered to both surfaces of the porous membrane 8 by adhering cells to one of the surfaces of the porous membrane 8 in a state where the one surface is located under a cell suspension, and by adhering cells to the other surface of the porous membrane 8 in a state where the other surface is located under a cell suspension.

### Example 2

FIGS. 2A and 2B show Example 2 according to the present invention. More specifically, FIG. 2A shows one surface of an upper substrate 14 in which a flow path 20 is provided and FIG. 2B shows one surface of a lower substrate 6 in which a flow path 12 is provided. The lower substrate 6 is the same as the substrate 6 of Example 1 shown in FIG. 1. The upper substrate 14 is different from the substrate 4 of Example 1 shown in FIG. 1 in that the flow path 20 is divided into two inlet flow paths 20-1 and 20-2 on its inlet side. One end of the inlet flow path 20-1 is connected to a liquid inlet 20-1a and one end of the inlet flow path 20-2 is connected to a liquid inlet 20-2a. The liquid inlets 20-1a and 20-2a are through holes.

The inlet flow path 20-1 has the same width and depth as the flow path 20, and the inlet flow path 20-2 has the same depth as the inlet flow path 20-1 but is narrower than the inlet flow path 20-1.

The structure of Example 2 is the same as that of Example 1 shown in FIG. 1 except for the flow path 20 and its liquid inlets 20-1a and 20-2a provided in the upper substrate 14.

As described above, Example 2 shown in FIGS. 2A and 2B has two liquid inlets, that is, the liquid inlet 20-1a connected to the wide inlet flow path 20-1 and the liquid inlet 20-2a connected to the narrow inlet flow path 20-2. The liquid inlet 20-1a is used to introduce a cell suspension to immobilize cells on the porous membrane. The liquid inlet 20-2a is used to introduce a reagent used in use of the biodevice.

### Example 3

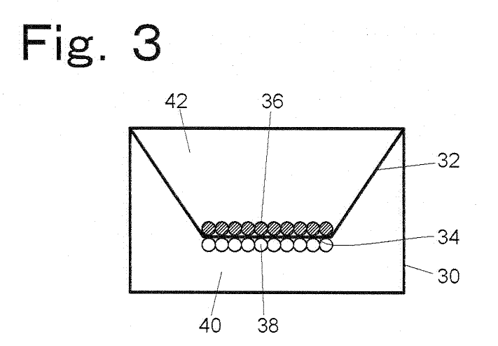

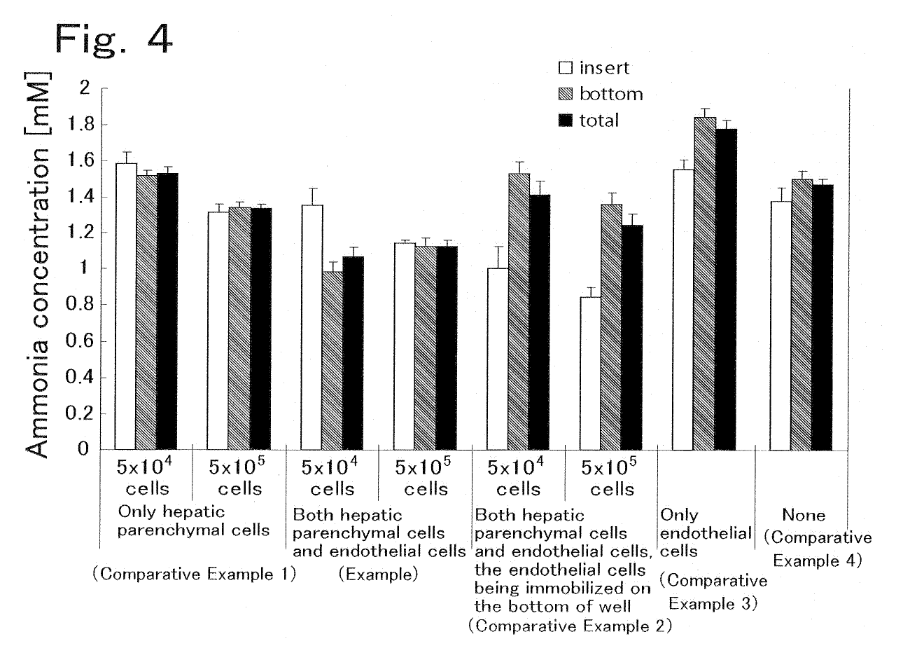

In order to verify the superiority of the structure proposed in the present invention, the following experiment was performed using a reaction container according to Example 3 shown in FIG. 3. As shown in FIG. 3, the reaction container includes a cylindrical well 30 having an upper opening with a bottom surface and an insert 32 detachably fitted into the opening of the well 30. The insert 32 is in the form of an inverted frustum of a cone whose diameter is decreased from its upper opening toward its bottom surface. The bottom surface is a circular porous membrane 34 having a diameter of 6.4 mm. The membrane 34 is a PET track-etched porous membrane having a thickness of 10 to 20 μm. Such a track-etched porous membrane has a plurality of micro-through-holes.

In the reaction container according to Example 3, about 5×10⁴ cells of human hepatoma cell line HepG2 were inoculated as hepatic parenchymal cells 36 onto one of the surfaces of the membrane 34 located inside the insert 32 (i.e., in FIG. 3, the upper surface of the membrane 34), and endothelial cells 38 were inoculated onto the other surface of the membrane 34.

Further, the following Comparative Examples 1 to 4 were prepared for the purpose of comparison with Example 3.

### Comparative Example 1

A reaction container was prepared in the same manner as in Example 3, except that inoculation of the endothelial cells 38 onto the other surface of the membrane 34 was omitted.

### Comparative Example 2

A reaction container was prepared in the same manner as in Example 3, except that the endothelial cells 38 were inoculated onto the bottom surface of the well 30 instead of the other surface of the membrane 34.

### Comparative Example 3

A reaction container was prepared in the same manner as in Example 3, except that inoculation of the hepatic parenchymal cells 36 onto one of the surfaces of the membrane 34 was omitted.

### Comparative Example 4

A reaction container in which cells were not inoculated onto either surface of the membrane 34 was prepared.

In order to determine whether there was a difference in the ability to metabolize ammonia among these reaction containers according to Example 3 and Comparative Examples 1 to 4, 700 μL of a medium 40 containing 2.0 mM-NH₄Cl was placed in the well 30 and 200 μL of a medium 42 containing no ammonia was placed in the insert 32 of each of the reaction containers. That is, in the case of, for example, the reaction container according to Example 3, the hepatic parenchymal cells 36 were brought into contact with the medium 42 containing no ammonia and the endothelial cells 38 were brought into contact with the medium 40 containing NH₄Cl.

The reaction containers according to Example 3 and Comparative Examples 1 to 4 containing the media 40 and 42 were incubated for 6 hours to measure a change in the ammonia content of each of the media 40 and 42. The measurement results are shown in FIG. 4. In the bar graph shown in FIG. 4, the values “5×10⁴” and “5×10⁵” under bars represent the number of hepatic parenchymal cells 36 inoculated onto one of the surfaces of the membrane 34. If the space under the bars is blank, it means that the hepatic parenchymal cells 36 were not inoculated.

In FIG. 4, each set of three bars is shown for each number of the hepatic parenchymal cells of the reaction containers. In each set of three bars, the leftmost bar indicated as “insert” represents the concentration of ammonia in the medium 42 (not containing ammonia before incubation) contained in the insert 32, the middle bar indicated as “bottom” represents the concentration of ammonia in the medium 40 (containing 2.0 mM ammonia before incubation) contained in the well 30, and the rightmost bar indicated as “total” represents the total concentration of ammonia in the medium 42 contained in the insert 32 and the medium 40 contained in the well 30.

In Example 3, the medium 42 initially containing no ammonia and brought into contact with the hepatic parenchymal cells 36 inoculated onto one of the surfaces of the membrane 34 corresponds to bile, and the medium 40 initially containing ammonia and brought into contact with the endothelial cells 38 inoculated onto the other surface of the membrane 34 corresponds to blood.

In Comparative Example 4, neither the hepatic parenchymal cells 36 nor the endothelial cells 38 were inoculated. Therefore, the result of Comparative Example 4 can be regarded as the result of the reaction container not having the ability to metabolize ammonia. If the reaction containers according to Example 3 and Comparative Examples 1 to 3 perform liver functions, it can be expected that the concentration of ammonia in the medium 40 having a high initial ammonia, concentration and contained in the well 30 will be decreased by ammonia decomposition and that ammonia will be transferred from the medium 40 contained in the well 30 to the medium 42 contained in the insert 32.

As can be seen from the results shown in FIG. 4, in the case of Example 3 (indicated as “Example” in FIG. 4), the total amount of ammonia was reduced. From the result, it can be estimated that decomposition of ammonia was carried out in the reaction container according to Example 3. Further, in the case of the reaction container according to Example 3 whose number of the hepatic parenchymal cells 36 inoculated onto one of the surfaces of the membrane 34 was 5×10⁴, the concentration of ammonia in the medium 40 contained in the well 30 was reduced but the concentration of ammonia in the medium 42 contained in the insert 32 was increased. From the result, it can be estimated that the phenomenon of transfer of ammonia from the medium 40 to the medium 42 occurred in the reaction container according to Example 3, that is, the reaction container according to Example 3 performed liver functions such as decomposition of ammonia and transfer of ammonia from blood to bile. On the other hand, the reaction containers according to Comparative Examples 1 to 3 did not perform liver functions.

It is to be noted that the materials and dimensions of the reaction containers according to Examples 1 to 3 are merely examples for experiment and are not intended to limit the present invention.

According to the present invention, it is possible to provide a biodevice that can be used not only as an artificial organ such as an artificial liver but also as a reaction container for use in drug metabolism tests using cells.

### DESCRIPTION OF THE REFERENCE NUMERALS

2 base body
4, 6, 14 substrate
8 porous membrane
10, 12, 20 flow path
34 membrane
36 hepatic parenchymal cell
38 endothelial cell

1. A method for producing a biodevice using a chip structure, the chip structure comprising:
a porous membrane provided in a base body and having two surfaces,
a first reaction chamber formed by the base body as a flow path which has a liquid inlet and a liquid outlet and through which a solution is allowed to flow along one of the surfaces of the porous membrane, and
a second reaction chamber formed by the base body as a flow path which has a liquid inlet and a liquid outlet different from the liquid inlet and the liquid outlet of the first reaction chamber and through which a solution is allowed to flow along the other surface of the porous membrane, the second reaction chamber being opposed to the first reaction chamber with the porous membrane being interposed between them,
the method comprising the steps of:
immobilizing first cells on the porous membrane in the first reaction chamber by introducing a suspension of the first cells through the liquid inlet of the first reaction chamber and then by allowing the chip structure to stand in a state where the porous membrane is located tinder the suspension; and
immobilizing second cells different from the first cells on the porous membrane in the second reaction chamber by introducing a suspension of the second cells through the liquid inlet of the second reaction chamber and then by allowing the chip structure to stand in a state where the porous membrane is located under the suspension.

2. (canceled)

3. The method for producing a biodevice according to claim 1,
wherein the flow paths corresponding to the first and second reaction chambers have a depth of 1 mm or less.

4. The method for producing a biodevice according to claim 1,
wherein the surfaces of the porous membrane on which the cells are immobilized are covered with a coating for cell culture.

5. The method for producing a biodevice according to claim 1, the biodevice being an artificial organ having hepatic parenchymal cells as the first cells and endothelial cells as the second cells.
